(19) United States
(12) Patent Application Publication
(10) Pub. No.: US 2009/0036867 A1
(43) Pub. Date: Feb. 5, 2009

(54) Medication Delivery Device Applying A Collapsible Reservoir

(75) Inventors: Kristian Glejboel, Glostrup (DK); Frank Daniel Mersebach, Hellerup (DK); Michael Hansen Svendsmark, Frederiksberg C (DK); Niels-Aage Hansen, Havdrup (DK); Lasse Hansen, Broendby Strand (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/159,799

(22) PCT Filed: Jan. 5, 2007
(86) PCT No.: PCT/EP2007/050119

§ 371 Date: Sep. 17, 2008

Related U.S. Application Data
(60) Provisional application No. 60/760,569, filed on Jan. 20, 2006.

(30) Foreign Application Priority Data
Jan. 6, 2006 (EP) 06388002.5

(51) Int. Cl. A61M 5/20

(52) U.S. Cl. 604/500; 604/151

(57) ABSTRACT

The present invention relates to a medication delivery device comprising pump means and a reservoir adapted for containing a medicament. The pump means is adapted to transfer the medicament from the reservoir to an outlet arrangement, said outlet arrangement being adapted to be operatively connected to an associated hypodermic needle. The pumps means is adapted to deliver a set dose of medicament during one or more pump strokes, wherein the stroke volume(s) of said one or more pump strokes is variable. The present invention further relates to a method for adjusting stroke volumes to a set dose of medicament.

### FIELD OF THE INVENTION

The present invention relates to a medication delivery device for delivering a liquid medicament from a collapsible reservoir to the human body. The medication delivery device employs a displacement pump where the displacement stroke volume can be adjusted. The present invention further relates to a collapsible reservoir for containing a medicament.

### BACKGROUND OF THE INVENTION

A prior art medication delivery device is disclosed in US 2002/007154. In the medication delivery device according to US 2002/007154 a liquid medicament is stored in a glass cartridge closed in the one end with a piston. Typically 3 ml. of a liquid medicament is stored in such glass cartridge. Furthermore, the medication delivery device of US 2002/007154 comprises a piston rod, acting on the piston, having a length sufficient to press the entire content of the glass cartridge out through a conduit mounted on the distal end of the medication delivery device. As disclosed in US 2002/007154 the piston rod is bendable in order to shorten the over all length of the medication delivery device, this bending however adds to the width of the delivery device.

Although one of the most refined medication delivery devices known in the art is the delivery device disclosed in US 2002/007154 it has nevertheless a number of important drawbacks.

A major drawback of a conventional medication delivery device of a type similar to the one disclosed in US 2002/007154 is that the only viable way for precise dosing of medicament is by controlled mechanical displacement of the piston. The displacement has to be extremely well controlled due to the large area of the dosing piston as even minute deviations from ideal piston position may result in either overdosing or under dosing of medicament. Another drawback of conventional medication delivery devices is that the length of the piston rod for expelling medicament from a cartridge needs to at least match the length of the cartridge containing the medicament. Thus, the length of the piston rod essentially dictates the overall length of the medication delivery device.

Another complicating problem with the above-mentioned type of medication delivery device is that comparably high forces are needed to displace the piston. Thus, high mechanical demands are put on the mechanical actuation system.

As a result of the need of a long piston rod and the high mechanical demands put on the mechanical actuation system delivery devices similar to the one described in US 2002/007154 tend to be rather bulky.

Smaller and more handy dosing systems are disclosed in the literature. A good example on a compact and portable system is given in WO 03/099358. Although small, the device disclosed in WO 03/099358 is a pre-filled single use auto injector. One important limitation of the system disclosed in WO 03/099358 is that it lacks the dose setting flexibility often required in modern therapy. If e.g. the medicament is needed for treatment of diabetes mellitus a wide range of doses is needed. The exact dose to be delivered to a patient depends among other things on the recent carbohydrate intake and on the amount of recent exercise. Thus, efficient treating of a disease like diabetes mellitus requires that the device can deliver a range of doses.

A great number of different dosing systems are described in the literature. Among these many of the described systems are small and convenient to use while others are precise and offers the flexibility required for treatment of complex diseases like diabetes mellitus. At the time of writing there is, however, a need for small, precise dosing systems that are at the same time simple and convenient to use.

It is an object of the present invention to provide a novel strategy for dosing systems having the virtues of advanced devices as described in 2002/007154 but the size and convenience of the simple devices as exemplified by WO 03/099358.

It is a further object of the present invention to provide a delivery device from which set doses can be administered said device being smaller and lighter compared to the present state of the art.

It is a still further object of the present invention to provide delivery means for medicament with a higher dose precision compared to any commercial available device.

It is a still further object of the present invention to provide a method for making delivery devices which are potentially more precise than conventional devices based on dosing from a glass cartridge.

### SUMMARY OF THE INVENTION

The above-mentioned objects and other objects are complied with by substituting the glass cartridge normally employed in flexible dosing systems with a collapsible reservoir special in that the pressure difference between the inside of the reservoir and the ambient is very small. This reservoir may be combined with a displacement pump where the displacement stroke volume can be adjusted according to the needs of the user.

A very precise and light dosing system can be obtained based on a collapsible reservoir and a displacement pump having an adjustable displacement volume. The most important virtue of a collapsible reservoir is in this context its pressure neutrality, i.e. the pressure inside the reservoir is approximately the same as the pressure outside the reservoir. Other important virtues over normal non-collapsible glass cartridge based reservoirs are low weight, compactness and low manufacturing costs. By taking advantage of the pressure neutrality it is possible to employ a displacement pump having an adjustable displacement volume to deliver very precise doses of medicament.

If a displacement pump is to be applied on a standard glass cartridge in a reliable manner, a force must be applied to the piston in order to overcome the erratic friction between the piston and the glass vessel. This would imply a permanently pressurised cartridge as the friction between the piston and the glass vessel is highly variable. However, a pressurised cartridge is not acceptable as failure of the pump may result in overdosing of medicament.

By avoiding the traditional piston rod and drive mechanism great simplification can be gained in the design of the medication delivery device.

If the medication delivery device is to be operated by electromechanical means the number of strokes required to deliver a given dose can be chosen randomly at no cost in the complexity of the system.

If the medication delivery device is to be operated manually or by a simple spring actuated mechanisms, the operation of the device is highly simplified if the complete dose of medicament is measured and delivered in a single stroke cycle. If the mechanical design of the device is such that only a single stroke of medicament can be delivered this furthermore improves the safety of the device significantly since multiple doses can not be delivered due to mechanical or electrical malfunctions.

By making a part of the collapsible reservoir from for example a sheet-like material it is possible to make a reservoir that is easily collapsible if the pressure outside the reservoir exceeds the inner pressure of the reservoir. As explained in details later, the term “collapsible” is not limited to reservoirs where the outer surface can collapse. This definition does also apply to a reservoir comprising a rigid outer shell but having an inner collapsible membrane made from a sheet-like material.

If a very simple, cheap and robust medication delivery device is wanted direct actuation of the pumping means may be the best choice. Direct actuation would typically be the preferred option for a third world device or a device containing a critical lifesaving drug. Direct actuation of the pumping unit may furthermore be an option if the mechanical actuation of the pumping unit fails.

Thus, in a first aspect the present invention relates to a medication delivery device for delivering a medicament, the medication delivery device comprising
pump means, and
a collapsible reservoir adapted for containing a medicament, the pump means being adapted to transfer the medicament from the collapsible reservoir to an outlet arrangement, said outlet arrangement being adapted to be operatively connected to an associated hypodermic needle,
   wherein the pumps means is adapted to deliver a set dose of medicament during one or more pump strokes, the stroke volume(s) of said one or more pump strokes being variable.

In the present content the term “collapsible” should be interpreted broadly. Thus, collapsible is to cover a reservoir comprising a flexible sheet-like material which changes its form with changes of the volume of the reservoir. In addition, the term collapsible is also to cover any arrangement which allows changes in a volume of a reservoir. Such changes in volume could be provided by moveable wall portions of the reservoir as long as the pressure inside the reservoir maintains at approximately the same level as the pressure outside the reservoir.

In the context of the present invention, “hypodermic needle” should be interpreted broadly, i.e. comprising injection needles, infusion sets, micro-needle arrays or other suitable means for mechanically penetrating the dermis, hereby allowing for infusion of a substance.

A pressure difference of around 0.1 bar between the interior of the collapsible reservoir and the surroundings may be acceptable. However, it is an advantage of the present invention that the interior pressure in the collapsible reservoir is kept at essentially the same level—independent of the amount of medicament in the collapsible reservoir.

The medication contained in the collapsible reservoir may in principle be any kind of medication, such as one or more peptides, one or more proteins or a combination hereof. Thus, the peptides or proteins may comprise insulin, insulin analogues, GLP or GLP analogues or a mixture comprising one or more of these.

The pump means and the collapsible reservoir may be rigidly arranged relative to each other. Such a rigidly arrangement between reservoir and pump means may be established by attaching at least part of the reservoir directly to a part of the pump means. The pump means and the collapsible reservoir may be arranged within an at least partly closed shell or housing. Openings allowing setting of a dose to be expelled may be provided.

The collapsible reservoir may comprise a substantially rigid portion and a collapsible portion, the collapsible portion being adapted to collapse into at least part of the substantially rigid portion upon changing the volume of the collapsible reservoir. Part of an inner surface of the collapsible portion of the collapsible reservoir may comprise a sheet material. A more detailed description of the required properties of such sheet material is given below.

The medicament contained in the collapsible reservoir may be sucked out of the reservoir applying displacement pump means. A displacement stroke and/or a restoring stroke of a pump cycle may at least partly be actuated by the user of the medication delivery device. Thus, the appliance of a force by the user of the medication delivery device may at least partly be utilized to expel the medicament from the stroke volume. Alternatively, or in addition, the displacement stroke and/or a restoring stroke of a pump cycle may at least partly be actuated by a spring mechanism. This spring mechanism may comprise a torsion spring, a linear spring or a combination thereof. Finally, the displacement stroke and/or a restoring stroke of a pump cycle may at least partly be actuated by an electromechanical actuator being controlled by an electronic control circuit comprising a microprocessor.

The medication delivery device according to the present invention may further comprise dose counting means which allows the user of the medication delivery device to set the dose of medicament to be expelled. The medication delivery device may further comprise end of content indicating means which informs the user of the medication delivery device that the collapsible reservoir is empty, or close to being empty, and, thus, needs to be replaced.

The medication delivery device may further comprise means for assisting the user of the medication delivery device deciding on the proper dose of medication. This assisting means may at least partly form part of a module, said module being adapted to be secured to the medication delivery device. A control unit may further be provided. The control unit may be adapted to communicate with the medication delivery device and/or with the module secured thereto.

The medication delivery device may further comprise at least one display member, said display member being arranged on the medication delivery device, on the module being adapted to be secured to the medication delivery device or as part of the control unit adapted to communicate with for example the medication delivery device.

The medication delivery device, the module adapted to be secured to the medication delivery device or the control unit may further comprise at least one microcontroller arranged in the medication delivery device, the attached module or in the control unit. The microcontroller may facilitate that dose information is fed to electromechanical means for controlling the delivered dose.

In order to power the medication delivery device according to the present invention the device may comprise power supplying means, such as a battery. Finally, the medication delivery device may be equipped with a hypodermic needle.

In a second aspect, the present invention relates to a method for delivering a set dose of medicament from a medication delivery device, the method comprising the steps of
setting the dose of medicament to be expelled from the medication delivery device, and
expelling the set dose of medicament from a collapsible reservoir by operating pump means of the medication delivery device in a manner so that the set dose of medicament is expelled using one or more pump strokes, wherein the stroke volume or stroke volumes associated with the one or more pump strokes is/are set in accordance with the dose of medicament to be expelled.

Thus, according to the present invention the stroke volume is set in accordance to the dose of medicament to be expelled. This implies that the stroke volume may be set to expel the set dose in a single pump stroke, or in a series of pump strokes with potentially different stroke volumes. Thus, a maximum of ten, eight, six, four or two pump strokes may be applied to expel the set dose of medicament. As already mentioned a single pump stroke may also be applied to expel the complete dose.

Similarly, a set dose of medicament may be expelled using a first pump stroke having a first stroke volume, said first pump stroke being followed by a second pump stroke having a second stroke volume, wherein the first stroke volume is different from the second stroke volume. The first and second stroke volumes may also be equal, as well as the number of applied pump strokes may differ from two.

In a third aspect, the present invention relates to a reservoir for medication delivery devices and for containing a medicament, the reservoir comprising a substantially rigid portion and a collapsible portion wherein at least a part of the rigid portion and at least a part of the collapsible portion are adapted to contact the medicament to be contained in the reservoir and wherein the collapsible portion is adapted to collapse into at least part of the substantially rigid portion upon expelling medicament from the reservoir.

As already mentioned the term “collapsible” should be interpreted broadly. Thus, collapsible is to cover a reservoir comprising a flexible sheet-like material which changes its form with changes of the volume of the reservoir. In addition, the term collapsible is also to cover any arrangement which allows changes in a volume of a reservoir. Such changes in volume could be provided by moveable wall portions of the reservoir as long as the pressure inside the reservoir maintains at approximately the same level as the pressure outside the reservoir.

The sheet-like material may comprise a sheet comprising a thermoplastic material which may form part of a multilayer sheet structure. The sheet material may further comprise one or more barrier layers. The sheet material may have a thickness smaller than 1 mm, such as smaller than 0.8 mm, such as smaller than 0.5 mm, such as smaller than 0.3 mm.

### BRIEF DESCRIPTION OF THE DRAWINGS

The present invention will now be explained in further details with reference to the accompanying figures, wherein

FIG. 1 shows an example of a possible layout of the main working parts of mechanics of a dosing device according to this invention,

FIG. 2 shows in details the sequence by which the actuator springs are energised and relaxed in a mechanism similar to the one depicted in FIG. 1,

FIG. 3 shows the simplest possible collapsible reservoir made from welded sheet material,

FIG. 4 shows how a collapsible reservoir can be included as a structural part of a dosing device,

FIG. 5 shows an example of how channels can be realised between the collapsible reservoir, the pumping unit and the outlet of a dosing unit,

FIG. 6 shows the simplest imaginable device according to the present invention,

FIG. 7 shows how an advanced motorized device could be realised according to the present invention,

FIG. 8 exemplifies how a flexible part may be used to seal the medication reservoir after filling,

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION OF THE INVENTION

In its most general aspect the present invention relates to a medication delivery device comprising some sort of displacement pump having an adjustable pumping stroke volume. This arrangement facilitates that the medication delivery device is capable of expelling an arbitrary preset dose of medication by applying one or more adjusted pump strokes. As the delivered dose depends not only on the number of pumping strokes but also on the chosen stroke volume very high dose precision can be obtained using one or few pumping strokes.

Thus, it is an advantage of the medication delivery device according to the present invention that the delivered dose is not proportional to the number of pumping strokes as the stroke volume of the pump arrangement can be adjusted to match a set dose of medication to be expelled.

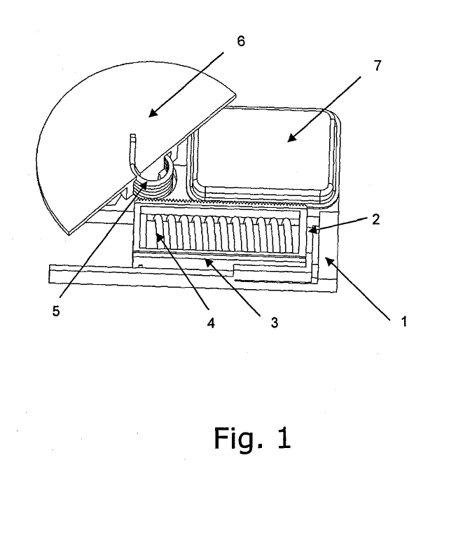

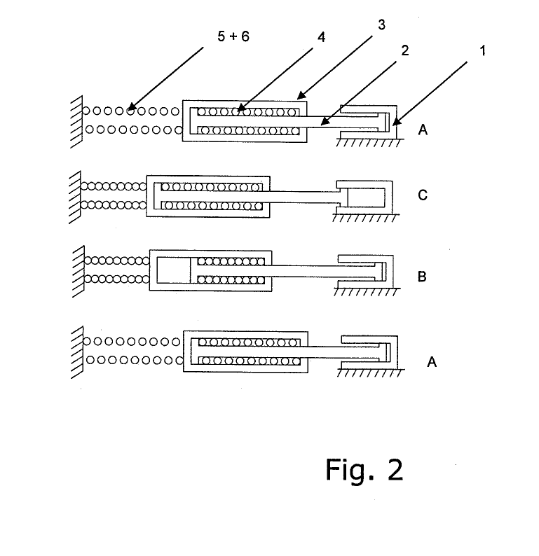

The function of an exemplary medication delivery device made according to the present invention will now be explained with reference to FIGS. 1 and 2. Central to the medication delivery device is a collapsible reservoir 7. The collapsible reservoir can be made in a number of different ways all having in common that at least a part of the inner side of the reservoir is collapsible and that there is only a minor pressure difference between the inside of the reservoir and the surroundings. By collapsible is thus meant that the reservoir is capable of changing its volume by having a collapsible or flexible inner wall structure whereby the volume of the reservoir is changeable while keeping a minor pressure difference between the inside of the reservoir and the surroundings.

In FIG. 1 an embodiment of a medication delivery device is depicted. For clarity the detailed functioning of the pumping mechanism is shown in FIG. 2. The references numbers of FIGS. 1 and 2 refer to the same components.

The overall function of the medication delivery device is that medication is drawn from the collapsible reservoir 7 (only shown in FIG. 1) to the cylinder 1 by retraction of the piston 2. As the retraction of the piston can be adjusted the volume of medication measured to the cylinder 1 is adjustable. To deliver the medication measured to the cylinder 1 the piston 2 is repositioned to its original position. This functionality is in the depicted device achieved by following the following step—starting from the bottom of FIG. 2:

Position A: The neutral position of the device. The volume enclosed by the cylinder 1 and the piston is minimised.

Position A-B: The device is prepared for drawing medication from the collapsible reservoir 7 to the cylinder 1 by moving the slider 3. By movement of the slider 3 the suction spring 4 and the dosing spring 5 are energized.

Position B-C: When the slider 3 is in its position to the right of FIG. 2 the piston 2 is released and the piston retracts due to the action of the suction spring 4. Now medication is sucked into the volume defined by the cylinder 1 and the piston 2. Note that the dosing spring 5+6 in FIG. 2 has the same function as the torsion spring 5 and the cogged dosing wheel 6 in FIG. 1—i.e. energising the slider 3 during delivery of medication.

Position C-A: After having measured medication into the volume defined by the cylinder 1 and the piston 2 the medication device is ready to deliver the measured dose. The measured dose is delivered by releasing the dosing spring 5+6 such that the piston is repositioned to the neutral position, hereby forcing the medication out of the cylinder.

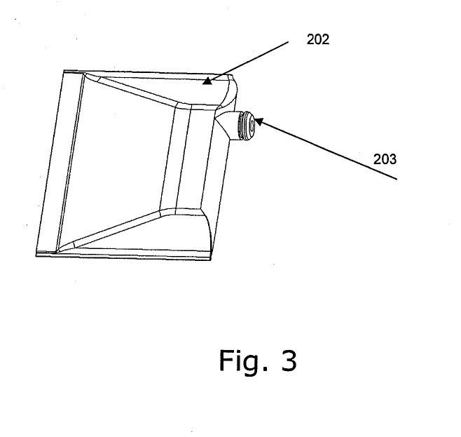

In its simplest form the reservoir is made from sheet material which is folded and welded, thus forming a closed bag (FIG. 3). If this type of collapsible reservoir 202 is employed it is normally necessary to attach some sort of coupling unit 203 to the reservoir. Although the sheet material for a simple reservoir can be chosen from a wide range of materials, the preferred materials are thermoplastics or laminates containing at least one layer of thermoplastic material. The sheet material should fulfil a number of different demands if employed for production of a reservoir as depicted in FIG. 3. Most important is that the reservoir should have excellent barrier properties and be compatible with the medication to be stored in the reservoir. Additionally, the material should be processable, i.e. if welding is chosen as the preferred process of joining the sheet material the material should be weldable. Additionally, the material should be able to withstand the mechanical loads to which it will be subjected during processing, transport and use. A final demand often put on the sheet material is that it should be possible to sterilize the material without critical degradation.

Due to the many conflicting demands on the sheet material, the sheet material may be a multilayer structure made from two or more layers having different properties. The sheet material will often be made predominantly from a laminate of multiple thermoplastic layers having the required mechanical properties. One or more barrier layers will be sandwiched between thermoplastic layers. Among inorganic barrier layers inorganic materials like Al AlOₓ, AlₓO_{y}N_{z}, SiOₓ, SiOₓN_{y}, SiNₓ are preferred. The numbers x, y, z does not refer to any specific stochiometric composition but rather indicate a range of numbers as barrier layers often are non-stochiometric substances. Among organic barrier layers polyvinylchloride (PVC), polyparylene, cyclo olefin copolymer (COC) polypropylene (PP) and polychlorotrifluoroethylene (PCTFE) are preferred materials. Among these PP, PVC, COC and PCTFE have a high mechanical strength. They may hence be used either in a laminate or as single layer sheets.

The sheet thickness strongly depends on the stiffness and barrier properties of the sheet material. In a preferred embodiment of the present invention the average thickness of the sheet material is less than 1 mm. In a more preferred embodiment of the invention the average thickness of the sheet material is less than 0.3 mm.

Depending on the properties of the sheet material a number of different strategies for joining may be employed, including adhesive bonding, welding and mechanical joining. Among these welding, preferably laser welding, RF welding or heat welding are preferred.

If a coupling unit (FIG. 3-203) is to be attached to the reservoir this coupling unit has to be made from a material which is compatible with the material of the reservoir. The coupling unit can either be a flexible rubber septum or a rigid coupling unit.

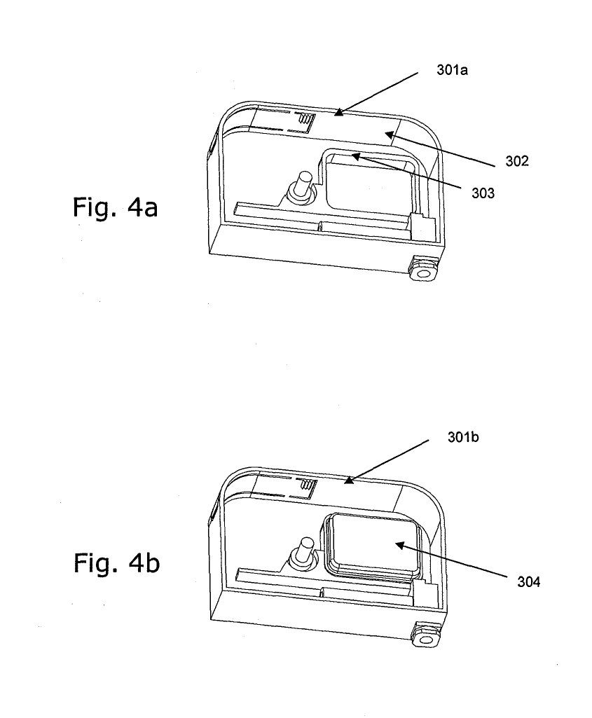

The use of fully collapsible reservoirs as illustrated in FIG. 3 may be beneficial for simple applications where the pumping unit is not fully integrated with the collapsible reservoirs. In an optimised system it may, however, be highly beneficial to integrate the reservoir completely with the dosing pump. An example of this is given in FIGS. 4a+4b.

In FIG. 4a a part of an injection moulded mechanism 301a is shown. In the mould injected piece a part of the pump 303 as well as a part of the collapsible reservoir 302 is integrated. According to this embodiment the collapsible reservoir is realised by joining a rigid part 301a+b to a collapsible part (FIG. 4b—304). Upon use the collapsible part of the reservoir 304 will collapse into the rigid counterpart 301. Thus, the reservoir will have pumping characteristics similar to the fully collapsible reservoir (FIG. 3) even though part of the reservoir is rigid.

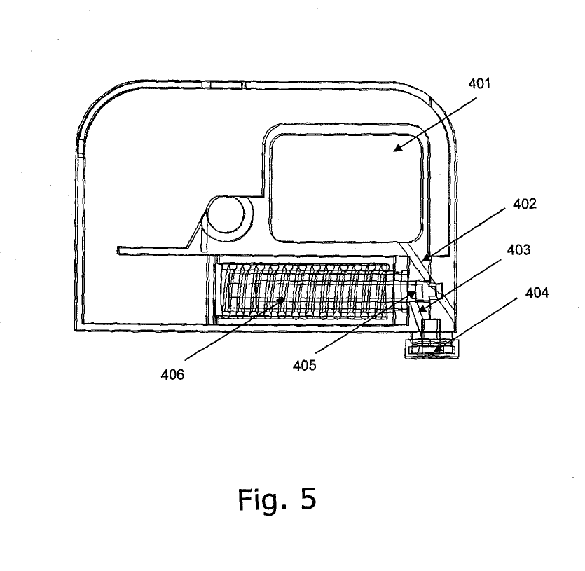

In FIG. 5 a cut through a device based on the mould injected part shown in FIG. 4 is shown. For clarity only parts related directly to the expelling of medication is included.

In this embodiment of the invention there is a closed channel or conduit 402 connecting the collapsible reservoir 401 and the pumping unit 405, 406. From the pumping unit there is an additional conduit 403 connecting the pumping unit 405, 406 to an outlet 404.

Upon use the device is prepared for injection by retraction of the piston 405 in the cylinder 406 hereby pulling the medication from the collapsible reservoir 401 into the cylinder 406. The device is now ready to deliver an amount of medication identical to the volume evacuated into the cylinder 406 by the withdrawal of the piston 405. Now a hypodermic needle is attached to the outlet 404 and the needle is inserted. Hereafter the piston is pushed back to its original position hereby forcing the medication stored in the cylinder to the outlet 404 where it runs to the needle.

In some embodiments, the outlet 404 is provided with a piercable septum for closing off conduit 403 during transfer of medication from reservoir 401 to cylinder 406. Said piercable septum is adapted to establish fluid communication when patient access means are attached to the outlet. Alternatively, or in addition, a valve arrangement can be arranged to provide fluid communication during discharge of medication from cylinder 406 and to provide termination of fluid flow through conduit 403 during transfer of medication from reservoir 401 to cylinder 406. Also, valve means may be adapted to control one-way fluid flow from reservoir 401 to cylinder 406.

One important advantage of a device having the dosing cylinder integrated with the reservoir containing medication is that the concentration of the medication and the piston area can be changed simultaneously. Thus, if the potency of a drug is changed and the area of the piston is changed accordingly, the stroke length of the piston will remain unaltered although the drug has been changed. If the device comprises a manually operated piston this is a very important comfort factor for the user as any change in use pattern is normally associated with uncertainty and discomfort.

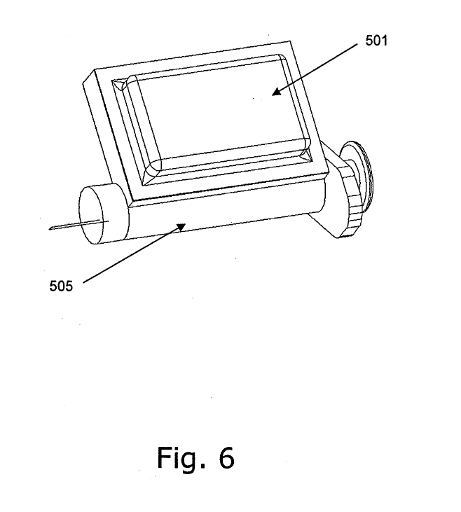

If a simple device or a very rugged device is wanted the pumping unit can be reduced to a fully manual operated mechanism reminiscent to a simple syringe. In FIG. 6 such a simple device is depicted. Note that the pumping unit 505 is fully integrated in the rigid part of the collapsible reservoir 501. Although simple the device depicted in FIG. 6 illustrates how the displacement of the piston rod visually communicates the remaining dose in the cylinder to the user. This feature may also be included in more advanced devices, either by making the displacement of the piston rod directly visible to the user or by including indicating means in the device which can communicate the remaining content in the reservoir to the user. Yet another desirable feature of the simple device compared to more automated devices is that the user knows that there is proportionality between the length the user moves the injection button during administration of the dose and the actual dose administered.

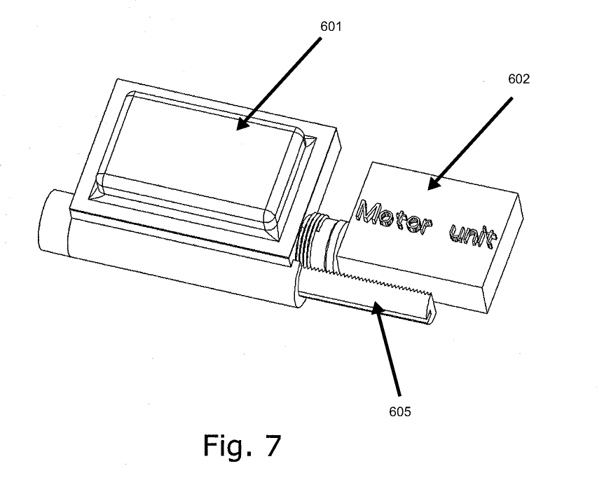

By minor modifications the simple rugged device shown in FIG. 6 can be modified to a highly advanced motor driven device. This is shown in FIG. 7. The most important difference in the dosing unit is that the simple piston is substituted by a toothed rod forming part of the piston 605 such that the piston 605 can be driven by the cogged wheel. Note that the motor 602 is only acting on a piston having a small diameter. Hence, dose precision can be obtained even with a motor unit with limited precision. If a unit like this is to be realised it would furthermore require a battery, a control unit and possibly a counter mounted directly on the cogged drive wheel.

A device made according to this embodiment may or may not include direct visual indications of a set dose, or direct visual indication of an expelled dose as found in the simple device. However, the same information may be presented in a graphic display.

One feature which might be beneficial to certain groups is the possibility to pre-program the device to give certain fixed amounts of medication. Although such a feature can be implemented in a purely mechanical device it is especially simple to implement a fixed-dose feature in a motor driven device.

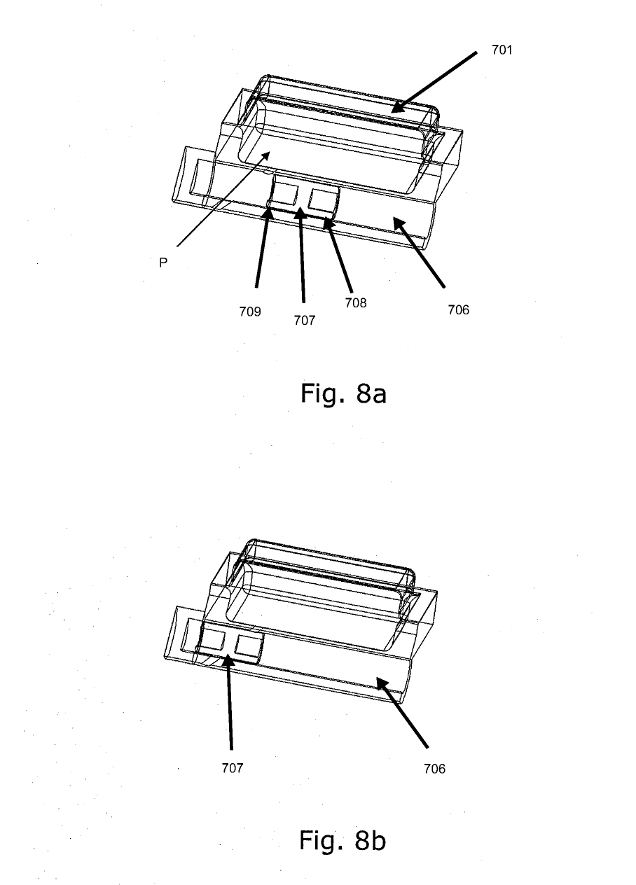

Different strategies are to be employed to fill collapsible reservoirs. If a pre-assembled semi-rigid reservoir of the type presented in FIGS. 1, 4, 5, 6 and 7 are to be employed one viable filling strategy is shown in FIGS. 8a+8b. As shown in FIG. 8a, a flexible plunger 707 is inserted into cylinder 706 prior to arranging the abovementioned piston into cylinder 706. After insertion of the plunger 707 the reservoir can be filled by inserting a filling needle parallel to depicted arrow P. By evacuating the reservoir 701 through the needle prior to filling, it is ensured that the reservoir can be completely filled with medication.

After the filling has been completed the plunger 707 is pressed to an end stop corresponding to the position shown in FIG. 8b, thus resulting in sealing of the reservoir. During subsequent use of the filled device, the plunger 707 remains in the position depicted in FIG. 8b.

By proper design of the plunger 707, it is possible to have it acting as a barrier during storage. Later when medication has to be drawn from the reservoir, the plunger has been designed such that the cylindrical lip portions 708 at the rightmost end of plunger 707 may collapse upon a negative pressure gradient from the cylinder towards the collapsible reservoir. Thus, upon drawing medication from the reservoir to the cylinder, the plunger acts like a single pass valve. Plunger 707 may be provided with a concave portion to receive a slideable piston (not shown) inserted into the right-hand side of cylinder 706.

In the embodiment shown in FIGS. 8a and 8b, and as discussed above, the device is provided with conduit means (not shown) for facilitating transfer of medicament from cylinder 706 to the outlet of the device. Such conduits means can be provided by arranging a fluid conduit (not shown) in the wall section of cylinder 406 thereby by-passing the plunger when sufficient pressure is applied on the slideable piston. A first end of such conduit is arranged to open into the left-hand side of the pump chamber, i.e. the pump chamber as defined by the cylinder 706 and the plunger 707 when plunger 707 is positioned in its left-most position (as shown in FIG. 8b). A second end of such conduit is arranged to open into cylinder 706 at the left-hand side of cylinder 706 so that the second end of the conduit is overlapped by a lip section 709 of plunger 707. Lip section 709 acts as a barrier when fluids pressure in the pump chamber is below a certain threshold. When fluid pressure inside the pump chamber exceeds this threshold, the lip section 709 of plunger 707 is adapted to collapse, thereby providing fluid communication across plunger 707.

Common to all parts in drug contact is that they should be compatible with the medication. The materials in contact with medication could be chosen from polyvinylchloride (PVC), cyclo olefin copolymer (COC), polyethylenethraphtalate (PET), polyethylene (PE), polyurethane (PU), polypropylene (PP), polychlorotrifluoroethylene (PCTFE), polyphenylsulphone (PPSU) and polyetherimide (PEI). Further materials for sealing's are needed. Among these thermoplastic elastomers based on PP, EPDM, SEBS or mixtures as well as liquid silicone rubber (LSR) are preferred. However the choice of materials is not limited to these.

Although a fully integrated reservoir has been used to exemplify the virtues of the collapsible reservoir it is also possible to implement a system where the collapsible reservoir is only an integral part of the dosing unit during use but not during manufacture and transportation. This embodiment of the invention is especially beneficial if the medication has a short shelf life, if different types of medication are to be delivered using the same mechanical device or if the pumping unit is reusable.

Evidently, the medication delivery device according to the present invention facilitates injection of in principle any fluid, solution or suspension containing any combination of therapeutic proteins and/or peptides. In a preferred embodiment the injected medication comprises insulin, insulin analogues, GLP or GLP analogues especially suitable for treatment of diabetes. In an equally preferred embodiment the injected medication comprises human growth hormones or human growth hormone analogues.

1. A medication delivery device for delivering a medicament, the medication delivery device comprising
pump means, and
a collapsible reservoir adapted for containing a medicament, the pump means being adapted to transfer the medicament from the collapsible reservoir to an outlet arrangement, said outlet arrangement being adapted to be operatively connected to an associated hypodermic needle,
wherein the pumps means is adapted to deliver a set dose of medicament during one or more pump strokes, the stroke volume(s) of said one or more pump strokes being variable.

2. A medication delivery device according to claim 1, wherein the pump means and the collapsible reservoir are rigidly arranged relative to each other.

3. A medication delivery device according to claim 1, wherein the pump means and the collapsible reservoir are arranged within an at least partly closed shell.

4. A medication delivery device according to claim 1, wherein the collapsible reservoir comprises a substantially rigid portion and a collapsible portion, the collapsible portion being adapted to collapse into at least part of the substantially rigid portion upon changing the volume of the collapsible reservoir.

5. A medication delivery device according to claim 4, wherein a part of an inner surface of the collapsible portion of the collapsible reservoir comprises a sheet material.

6. A medication delivery device according to claim 1, wherein a displacement stroke and/or a restoring stroke of a pump cycle are/is at least partly actuated by the user of the medication delivery device.

7. A medication delivery device according to claim 1, wherein a displacement stroke and/or a restoring stroke of a pump cycle are/is at least partly actuated by a spring mechanism.

8. A medication delivery device according to claim 1, wherein a displacement stroke and/or a restoring stroke of a pump cycle are/is at least partly actuated by an electromechanical actuator.

9. A medication delivery device according to claim 8, wherein the electromechanical actuator is adapted to be controlled by an electronic control circuit comprising a microprocessor.

10. A medication delivery device according to claim 1, further comprising dose counting means.

11. A medication delivery device according to claim 1, further comprising end of content indicating means.

12. A medication delivery device according claim 1, further comprising means for assisting the user of the medication delivery device deciding on the proper dose of medication.

13. A medication delivery device according to claim 1, further comprising power supplying means, such as a battery.

14. A medication delivery device according to claim 1, further comprising a hypodermic needle.

15. A method for delivering a set dose of medicament from a medication delivery device, the method comprising:
setting the dose of medicament to be expelled from the medication delivery device, and
expelling the set dose of medicament from a collapsible reservoir by operating pump means of the medication delivery device in a manner so that the set dose of medicament is expelled using one or more pump strokes, wherein the stroke volume or stroke volumes associated with the one or more pump strokes is/are set in accordance with the dose of medicament to be expelled.

16. A method according to claim 15, wherein a maximum often pump strokes are applied to expel the set dose of medicament.

17. A method according to claim 15, wherein the set dose of medicament is expelled using only a single pump stroke.

18. A method according to claim 15, wherein the set dose of medicament is expelled using a first pump stroke having a first stroke volume, said first pump stroke being followed by a second pump stroke having a second stroke volume, wherein the first stroke volume is different from the second stroke volume.

19. A reservoir for medication delivery devices and for containing a medicament, the reservoir comprising:
a substantially rigid portion, and
a collapsible portion,
wherein both rigid and collapsible portions are adapted to contact the medicament, the collapsible portion being adapted to collapse into at least part of the substantially rigid portion upon expelling medicament from the reservoir.

20. A reservoir according to claim 19, wherein the collapsible portion of the reservoir is at least partly formed by a sheet material.

21. A reservoir according to claim 20, wherein the sheet material comprises a thermoplastic material.

22. A reservoir according to claim 21, wherein the thermoplastic material forms part of a multilayer sheet structure.

23. A reservoir according to claim 21, wherein the sheet material further comprises one or more barrier layers.

24. A reservoir according to claim 20, wherein the sheet material has a thickness smaller than 1 mm, such as smaller than 0.8 mm, such as smaller than 0.5 mm, such as smaller than 0.3 mm.
